(19) United States
(12) Patent Application Publication
(10) Pub. No.: US 2007/0066956 A1
(43) Pub. Date: Mar. 22, 2007

(54) Systems and methods for entering temporary basal rate pattern in an infusion device

(75) Inventor: Evan Finkel, Northridge, CA (US)

Correspondence Address: PILLSBURY WINTHROP SHAW PITTMAN LLP, P.O BOX 10500, McLean, VA, 22102, US

(73) Assignee: Medtronic Minimed, Inc., Northridge, CA (US)

(21) Appl. No.: 11/190,804

(22) Filed: Jul. 27, 2005

(51) Int. Cl. A61M 31/00

(52) U.S. Cl. 604/500

(57) ABSTRACT

A system and method of temporarily adjusting a delivery of fluid or medications to a user from an infusion device that provides various delivery customizations, and may include a user interface. The user interface may provide access to a menu from which users can select specific delivery options appropriate for different times and situations.

### FIELD OF THE INVENTION

1. Field of the Invention

Embodiments of the invention relate generally to systems and methods for delivering fluids to an individual's body. More particularly, embodiments of the invention relate to an infusion device with a user interface and a system that allows a temporary change in the basal rate and/or pattern of the fluid that is delivered into the individual's body.

2. Description of Related Art

Infusion devices and systems are relatively well known in the medical arts for use in delivering or dispensing a prescribed medication such as insulin to a patient. In one form, such devices include a relatively compact housing adapted to receive a syringe or reservoir carrying a prescribed medication for administration to the patient through infusion tubing and an associated catheter or infusion set.

These infusion devices are commonly used for the programmed delivery of measured doses of an infusion formulation. The infusion device may include a small drive motor connected via a lead screw assembly for motor driven advancement of a reservoir piston to administer the medication to the user. Programmable controls can operate the drive motor continuously or at periodic intervals to obtain a closely controlled and accurate delivery of the medication over an extended period of time. Such infusion devices are used to administer insulin and other medications, with exemplary constructions being shown and described in U.S. Pat. Nos. 4,562,751; 4,678,408; 4,685,903; 5,080,653 and 5,097,122, which are incorporated by reference herein.

Infusion devices and systems generally include a display and an input device. In some infusion devices known in the art, the display and input device are part of the housing. In others, the display and input device are contained in a separate housing and may act as a remote commander. Thus, the entire infusion device, including any input and output devices, may be composed in a unitary housing or also be formed from two separate components.

An infusion device and system of the above type is described in U.S. Pat. No. 6,872,200. The infusion system includes an external infusion device and a remote commander that operate together to infuse a fluid into a body. The external infusion device includes a housing, a receiver, a processor and an indication device. The receiver receives remotely generated commands and controls the external infusion device in accordance with the commands. The remote commander includes a commander housing, a keypad for transmitting commands, and a transmitter for transmitting commands to the receiver of the external infusion device.

Infusion devices of the general type described have provided significant advantages and benefits with respect to accurate delivery of medications such as insulin over an extended period of time. The infusion device can be designed to be extremely compact as well as water resistant, and may thus be adapted to be carried by the user, for example, by a belt clip or the like. As a result, important medication can be delivered to the user with precision and in an automated manner, without significant restriction on the user's mobility or life style, including in some cases the ability to participate in water sports.

However, the amount and manner in which the fluid or medication can be delivered is generally limited to standard programs that do not provide many options to enter temporary delivery rates that change a current basal rate for a limited time period. As such, these standard programs do not provide convenient ways to adjust a user's basal rate in response to a temporary change in the user's daily schedule. There are a few infusion devices that allow temporary changes to current basal rates, such as U.S. Pat. No. 6,852,104, but these devices generally provide either a straight fixed or a percentage that is to apply a temporary basal rate change in terms of the current basal rate. Because the appropriate amount of medication such as insulin to be delivered is influenced by a wide range of factors, such as the user's weight, body type, food preferences and intake, exercise habits, the delivery dosages, a user's insulin needs fluctuate daily, depending on his or her schedule of events. Adjusting a current basal rate as a percentage or fixed amount may not be the best way to assist a user in maintaining their treatment regimen.

A concern with applying changes as percentages of current basal rates, however, is that the user may not be able to quickly or accurately calculate the actual dosage or value of insulin being delivered from the percentage. A user could miscalculate the actual dosage or value and thus input an inappropriate percentage of the current basal rate. Under or overdosing of medication can sometimes lead to dire consequences. Thus, although it is convenient to apply a temporary change in terms of a percentage of the current basal rate, it is important to enter temporary basal rates in such a manner with some form of a safety check.

### BRIEF SUMMARY OF THE INVENTION

In accordance with embodiments of the invention, a method and delivery system are provided that allow for the enhanced control of the delivery of a fluid or medication. An embodiment of the present invention includes an infusion device having a user interface that includes a display and input device. The user interface can be used to customize the delivery of medication, such as insulin, according to a user's pattern. For example, the user interface may allow a user to program various commands that can be set in the memory of the infusion device to control how and when the medication is to be delivered as a pattern. This pattern will store the basal rates needed at various times of the day or in accordance with a schedule of events. The user interface may further include control and display features that will provide additional choices on how the delivery rate can be temporarily changed to address any changes in the user's daily schedule. These features also provide visual calculations for the temporary basal rate selected for the convenience and safety of the user.

The user interface may be housed in the same or separate structure as the infusion device. In one embodiment, a remote commander may transmit the programmed commands through a transmitter to a receiver in the infusion device. In this manner, the user interface does not necessarily need to be carried with the infusion device, and can be carried separately for less bulk. A keypad may be a part of the user interface to facilitate the programming of commands to the infusion device. The transmitter may then facilitate the delivery of the customized dosages.

Further embodiments include a memory for storing programs, and a receiver also capable of receiving software updates and facilitating remote programming of the infusion device options to customize delivery of the medication to the user.

The user interface provides various options to customize the control of the rate that the medication is delivered into an individual's body. For example, the user may select to deliver the medication as a bolus, a constant rate, a basal rate pattern set in the device memory, a variable percentage of the basal rate pattern, or a fixed percentage of the current basal rate. The interface may further include a suspend function that allows the user to select periods of time for temporarily suspending medication delivery from the infusion device. In some embodiments of the invention, after a period of time for temporarily suspending medication delivery has been concluded, the infusion device automatically resumes medication delivery.

From the user interface, the user may choose pattern settings and customize medication delivery dosages. The infusion device may be set at a specific pattern according to certain schedules. The pattern is stored in the memory, and in the case that some expected or unexpected change occurs in the user's schedule, the user can alter the current basal rate accordingly by a difference in percentage. The user interface provides one or more options with which to temporarily change delivery rate in terms of percentages of current basal rates. The options provide user convenience in handling insulin needs, even when a change in a user's schedule necessitates a temporary change in the current basal rate. The user has the option to set a temporary basal rate type as a variable percentage or a fixed percentage. A variable percentage sets the temporary basal rate as a percentage of a varying basal pattern over a period of time. A fixed percentage sets the temporary basal rate as a percent of the current basal rate for a period of time. It does not vary but remains at that rate for the entire time period.

In yet another embodiment, the display of the user interface is adapted to calculate and show the actual basal rate value or dosage in units/hour after the user modifies a current medication delivery pattern. If a user selects a fixed percentage of the current basal rate for a specified time, the actual basal rate in units/hour that the percentage is equivalent to will be displayed on the screen for the specified time period. The user may then be prompted to select whether to proceed with the modification or to cancel. The calculation displayed on the screen serves as a precautionary measure to ensure the user is aware of his or her selection and that the selection is correct before the modification is sent to the infusion device. For example, if the user selects 50% of the current basal rate for one hour, at the moment the current basal rate is 20 units/hour, the screen will display “50% of the current basal rate is 10 units/hour.” A prompt such as “Proceed?” may then appear, either subsequently or on the same screen, asking the user to either confirm the percentage or to cancel.

In the alternative, if the user selects a variable percentage of the basal pattern, the screen may display what the actual rate in units/hour will be at each time point in which the current pattern changes. For example, if the user selects 50% of the basal pattern for one hour, where the current basal pattern delivers 10 units/hour for the first twenty minutes and then increases to 20 units/hour for the next forty minutes, the screen will display “50% of the current basal pattern for one hour is 5 units/hour for 0-20 minutes and 10 units for 21-60 minutes.” A prompt may also be used to ask the user to confirm or cancel the selection. The screen may display the actual rate in units/hour for other modifications selected, besides those described above, to prompt user confirmation before the command is transmitted to the infusion device.

In a related embodiment, there may be a block function that prevents delivery of medication after a potentially harmful amount of medication is requested by the user. For example, the block function can be used to prevent the delivery of an unusually large bolus, a bolus requested too soon after a previous bolus is delivered or a request for too low of a total medication dose. Such functions typically include a warning signal to the user that a potentially harmful amount of medication was requested. In the alternative, the block function may also be triggered in a situation where a medication dosage, that is to be delivered over a period of time, exceeds a target value.

### BRIEF DESCRIPTION OF THE DRAWINGS

A detailed description of embodiments of the invention will be made with reference to the accompanying drawings, wherein like numerals designate corresponding parts in the figures.

FIG. 1 is a block diagram of an exemplary infusion device according to an embodiment of the invention.

FIG. 2 is a block diagram of the infusion device configured through a remote communication station according to an embodiment of the invention.

FIG. 3 illustrates fixed and variable settings according to an embodiment of the invention.

FIG. 4 illustrates the user interface with a display of a calculated actual basal rate value or dosage in units/hour according to an embodiment of the invention.

FIG. 5(a) is a chart illustrating a temporary basal rate type of variable percentage applied to a basal rate pattern expressed in units per hour according to an embodiment of the invention.

FIG. 5(b) is a chart illustrating a temporary basal rate type of fixed percentage applied to a basal rate pattern expressed in units per hour according to an embodiment of the invention.

FIG. 6(a) illustrates a flow chart diagram of the new rate menu according to an embodiment of the invention.

FIG. 6(b) illustrates a flow chart diagram of the temporary basal rate menu according to an embodiment of the invention.

### DETAILED DESCRIPTION OF THE INVENTION

In the following description, reference is made to the accompanying drawings which form a part hereof and which illustrate several embodiments of the present inventions. It is understood that other embodiments may be utilized and structural and operational changes may be made without departing from the scope of the present inventions.

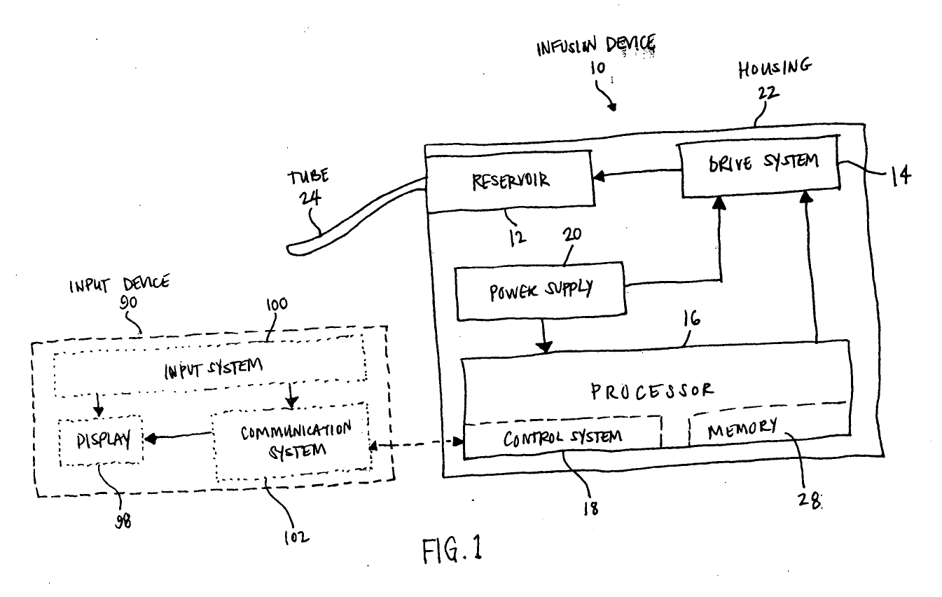

As shown in FIG. 1, an embodiment of the invention is an infusion device 10. The infusion device 10 includes a processor 16 that is contained in a housing 22 of the device 10. The processor 16 is coupled to internal memory 28 and can be used to run programs that control the infusion device 10 through a control system 18. The memory 28 stores the various programs as well as historical data, user defined information, settings and other parameters. The memory 28 can be used to store specific delivery patterns, such that the user may later select one or more stored delivery patterns to initiate specific delivery rates without having to input the rates and time periods each time. In alternative embodiments, the internal memory may be a flash memory. The memory 28 may also be a removable memory that is included in a flash memory card. Other memory devices known in the art may be used, for example, any volatile or non-volatile memory device. In one embodiment, the infusion device 10 can be programmed manually through an input device such as a keyboard or touch screen. The input device 90 includes a communication system 102 that may be coupled to the processor 16 of the infusion device 10. The communication system 102 sends the information entered into the input system 100 to the infusion device 10. The input device 90 may be separate from the infusion device or built directly into the device. The input device 90 may include a display 98 such as an organic light emitting diode (OLED) display, a light emitting diode (LED) display, and a liquid crystal display (LCD). Feedback from the infusion device 10 on the status or programming changes may be shown on the display 98.

The processor 16 is in communication with a medication or fluid reservoir 12 containing fluid that can be directed through an outlet tube 24 in the reservoir 12, and into a body of a user through tubing and an infusion set. The fluid is pumped by a drive system 14 that operates through a power supply 20. In other embodiments, the infusion device 10 can deliver the fluid directly into the user without tubing or an infusion set. For example, the infusion device can be located on or in the user's body at an infusion site.

In an illustrative embodiment of the invention, the infusion device can be a medication infusion device capable of delivering insulin to a diabetic user at a rate of about 0 to 35 units/hour in basal rates and up to about 25.0 units per meal bolus of U-100 insulin. The infusion device may also deliver other concentrations of insulin and/or other medications and may operate at other rates in further embodiments. Alternatively, the invention can deliver other fluid compositions such as saline, and fluids that include vitamins, peptides, hormones, proteins, enzymes, vaccines, and the like.

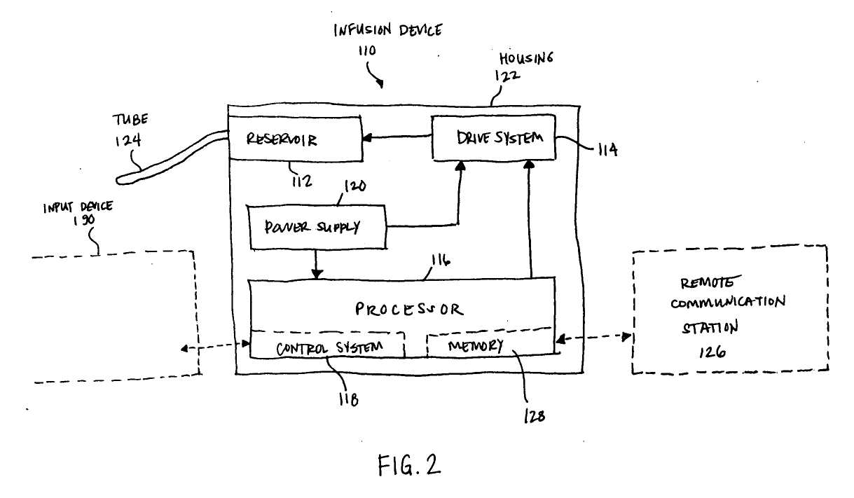

As shown in FIG. 2, operation of the infusion device 110 is typically directed through programming that can be derived from a variety of possible sources. The programming can be entered directly into the device 110 through an input device 190 or transferred to the processor 116 from a communication station 126, separate from the infusion device housing 122. FIG. 2 is a block diagram of an embodiment of the infusion device 110 configured through a communication station 126. The infusion device 110 may download stored information through the communication station 126. Moreover, the information, programs and data may be downloaded to a remote or local PC, laptop, communication station, or the like, for analysis and review by a physician or trained professional through a transceiver. The data may also be downloaded through the communication station 126 to a remote location over communication lines, such as by wired, modem, wireless connection or other electronic communication methods.

The information sent from the communication station 126 is transferred to the processor 116 of the device 110 and either stored in the memory 128 or used by the control system 118 to deliver the fluid or both. The power supply 120 then allows the drive system 114 to pump the fluid from the reservoir 112 through the outlet tube 124 and into the user's body.

In another embodiment, the infusion device may be remotely programmed, such as for example through a computer software program. The computer software program is essentially a virtual input device that includes the same commands and controls that a keyboard or touch screen would have in the previous embodiments. The infusion device processor may be synched with the computer program on a computer, so that changes made on the computer are indicated in the processor, and vice versa.

Additional software may be used on the computer for other functions, such as medication delivery, visual display of pattern history, etc. See U.S. Patent Publication No. US-2002-0193679A1 filed Jun. 26, 2002 and entitled “Communication Station and Software for Interfacing with an Infusion Pump, Analyte Monitor, Analyte Meter, or the like,” U.S. patent application Ser. No. 11/172,492 filed Jun. 29, 2005 and entitled “Flexible Prandial Glucose Analysis Using Varying Report Time,” and U.S. patent application Ser. No. 10/913,149 filed Aug. 6, 2004 and entitled “Medical Data Management System and Process”, which are all herein incorporated by reference in their entirety. The software may include graphing capabilities and spreadsheets and other data displays. In certain embodiments, the processor of the infusion device is configured to display the information from the software on the computer. For example, the processor may be configured to display on the computer screen a graph of medication delivery over a certain period of time, such as the basal delivery for the past 24 hours. The user may then choose to print out the graph from the computer.

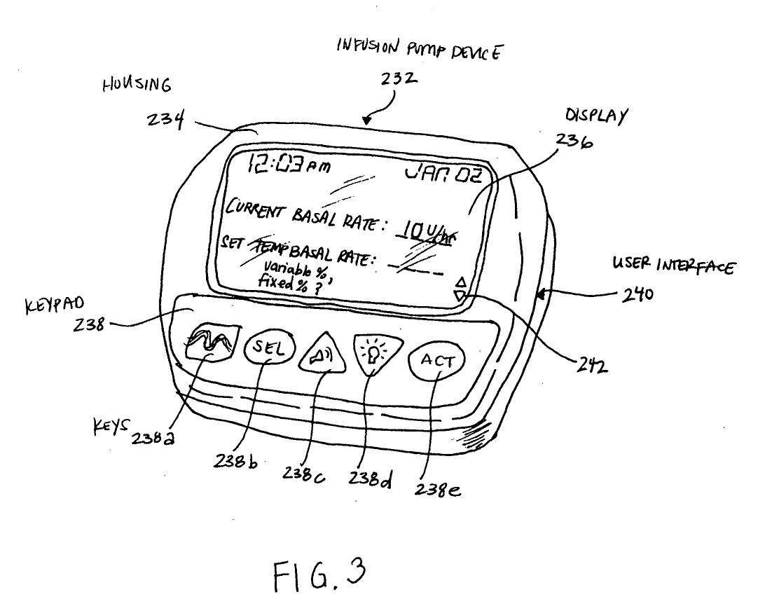

FIG. 3 shows an embodiment of the user interface 240 including a display 236 and keypad 238. The user interface 240 is positioned on the top of the housing 234 of the infusion device 232, but may be positioned elsewhere in different embodiments. The keypad 238 has keys 238a, 238b, 238c, 238d, 238e, to allow the user to input information. In alternative embodiments, other input devices, such as for example, buttons may be used. In FIG. 3, the current basal rate is shown at 10 units/hour. The user can select a temporary basal rate type to modify the current basal pattern by a percentage rather than entering an entirely new rate. In FIG. 3, the options provided are illustrated as a temporary basal rate type of variable percentage or a fixed percentage. Different patterns may be used for different days or created for different lengths of time. For example, patterns may be generated for weekdays and weekends as well as patterns for rest periods and exercise periods. In this manner, the patterns may be programmed accordingly to a known schedule of these events. The up-and down-arrow symbols 242 indicate that parameter values within each menu may be modified by pressing the UP key 238c or the DOWN key 238d.

Other display settings may be customizable, including, but not limited to, the background, sounds, fonts, and wallpaper. There may be a children's mode, with limited features available so that a child cannot dispense too much medication at once. Different display features may be included in the module and/or may be downloaded from a computer. There may also be a button or switch or other input to stop the pump in an emergency. To avoid the emergency stop from being activated accidentally, there may be a safety feature implemented.

There may be included menus accessible from the user interface for programming delivery patterns. One or more delivery patterns may be created and/or stored in the infusion device by using the various functions and options provided through the display on the user interface. The delivery patterns are composed of a series of setting options for each particular parameter to be programmed. The various pattern functions/options may be accessible from the display of FIG. 3. In one embodiment, there may be keys to depict the keystroke used to change from one menu to the next. Up-and down-arrow symbols may be used to indicate that parameter values within each menu may be modified by pressing the UP and DOWN keys. The user begins by moving an indicator from one end of the pattern description until it arrives at the parameter that the user wishes to modify. In the embodiment, the user can press a SEL key to select the parameter to be modified. Once that parameter is selected, the user may move the indicator up or down until the desired value is shown. After the parameters are set to the desired value, the user may accept the selections by pressing an ACT key. In embodiments, once the desired value is selected, the interface automatically applies the value to the subsequent parameters affected. Thus, all prior parameters are left unaffected, but all subsequent parameters are modified by the preceding parameter changed. All intervening parameters between two changed parameters will retain the setting of the first parameter. Alternatively, the user interface may be text based.

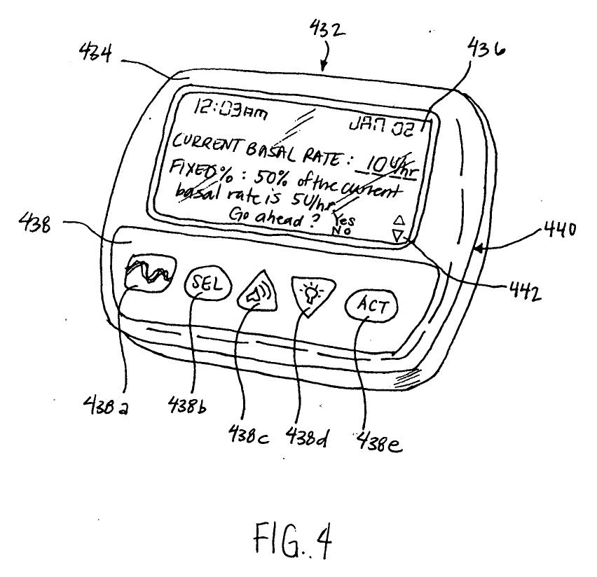

FIG. 4 illustrates an exemplary user interface 440, positioned on the top portion of the housing 434, with a display 436 of a calculated actual basal rate value or dosage in units/hour according to an embodiment of the invention. In the embodiment, a user enters the percentage that he or she wishes to modify either the current basal rate or the pattern by and the user interface will display the actual amount of insulin in units/hour, with a prompt that requires the user to execute a command or confirm to proceed. The user interface 440 provides various options that can customize the control of the rate that the medication is delivered from the infusion device 432 into an individual's body. In one embodiment, the user may use the keys 438a, 438b, 438c, 438d, 438e, of the keypad 438 to select the parameters to be set or modified. The up- and down-arrow symbols 442 may be used to select the desired values for the designated parameters. For example, the user may select to deliver the medication as a bolus, a constant rate, a basal pattern set in the device memory, a variable percentage of the basal pattern, or a fixed percentage of the current basal rate.

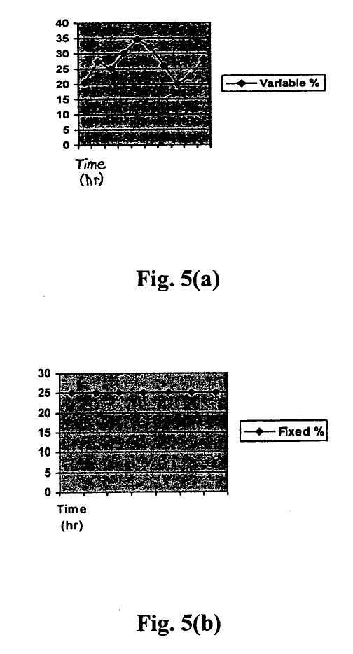

From the user interface, the user may choose various pattern settings and customize medication delivery dosages. The user can conveniently change a current basal rate by a percentage (e.g., to adjust the current basal rate in response to a change in the user's schedule). The percentage selected may be either variable or fixed. As shown in FIG. 5(a), a variable percentage sets the temporary basal rate as a percentage of the basal profile over a period of time, and applies the percentage change equally across the pattern, including any changes in rate that occur during the pattern. As shown in FIG. 5(b), a fixed percentage sets the temporary basal rate as a percentage of the current basal rate for a period of time. It does not vary but remains at that rate for the entire time period. Both these options conveniently allow a user to adjust the rate as a percentage of the current basal rate, rather than requiring the user to estimate and enter a new delivery rate. In using percentages, a user may more easily estimate how much insulin is needed in comparing the present situation to the degree of the change of the schedule.

In another embodiment, the option to set a temporary basal rate may be grouped under a command that is accessible from a setup menu, such as for example “temp basal rate.” The user may select this option and indicate whether the dosage desired is to be delivered as a variable percentage or a fixed percentage. In the alternative, once the user selects “temp basal rate,” the menu will prompt the user to choose on of the options, such as variable percentage or fixed percentage.

In yet another embodiment, the temporary basal rate may be selected from a number of pre-set percentages. For example, after the user selects the format that the temporary basal rate is to be delivered, a scroll-down window of percentages are displayed from which the user may select a percentage. The percentages may be presented in increments of 5 percent. In alternatives, the increments may be larger or smaller. There may also be included a setting that allows the user to choose the increments to be shown. In addition, if the user wishes to enter a specific percentage that is not shown in the pre-set choices, an option may be provided as “other percentages,” in which the specific percentage can be manually entered. Another embodiment may prompt the user to select the percentage prior to selecting the format in which the temporary basal rate will be delivered.

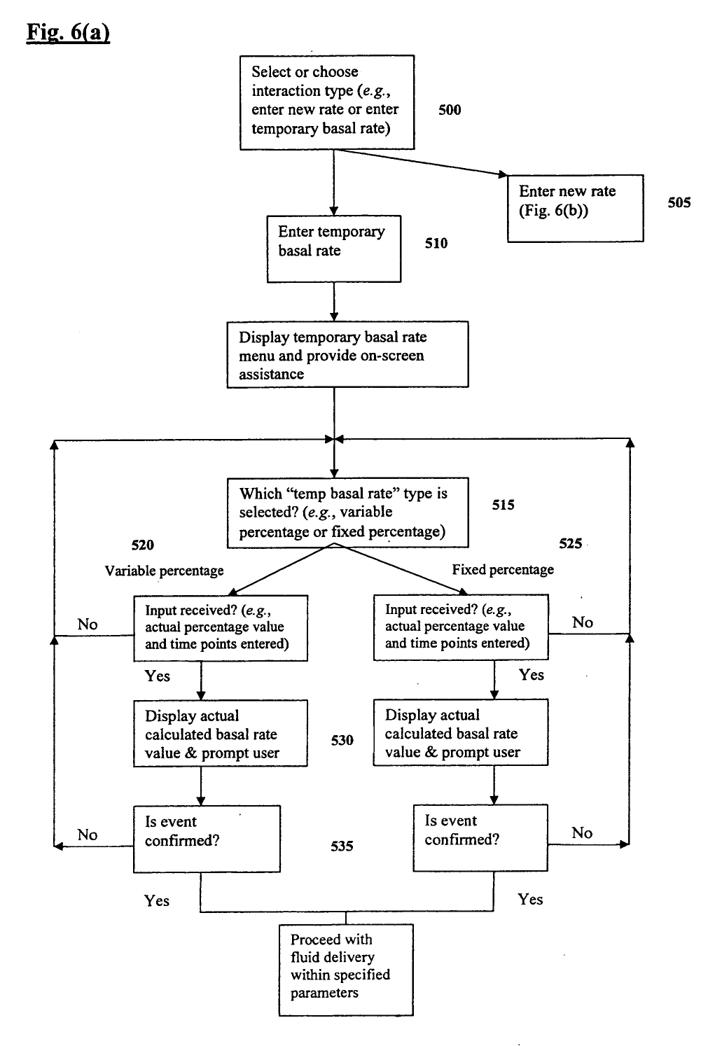

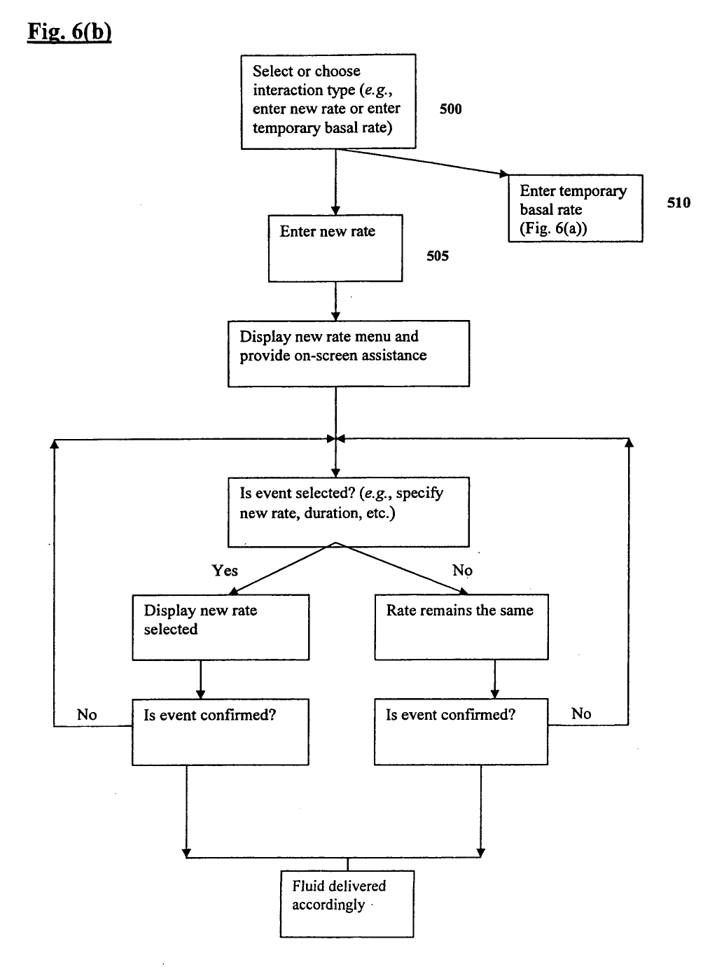

From the user interface, the user may access a setup menu 500 from which to select or choose interaction types, such as for example, pattern settings or customize medication delivery dosages, as shown in FIGS. 6(a) and 6(b). The menu may provide the option whether to enter a new rate 505 or a temporary basal rate 510. As seen in FIG. 6(b), the user may select an option 505 to enter a new delivery rate that will replace the current basal delivery rate for the amount of time specified. Alternatively, as seen in FIG. 6(a), the user also has the option 510 under “temp basal rate,” or other similar phrase, to select and enter a temporary basal rate of a the current basal rate. By selecting the option 510, the user may next select the temporary basal rate type as a variable percentage or a fixed percentage 515. A variable percentage 520 sets the temporary basal rate as a percentage of the basal pattern over a period of time. Where the basal rate varies over time, the variable percentage remains at a substantially uniform percentage of the basal rate as it varies. A fixed percentage 525 sets the temporary basal rate as a percentage of a constant value, the constant value being a current basal rate value of the basal rate at the instant the fixed percentage is selected. The rate does not vary but remains at that rate for the specified time period.

After choosing which option he wants, the user may later set the temporary basal rate. For example, if the user has chosen a variable percentage, he will be asked to choose a percent and to choose a period of time for the temporary basal rate. If the user chooses 50% and 1 hour, the basal rate will lower to 50% of the basal pattern for the entire 1 hour period. It will vary according to the original basal pattern. For example, if the original basal pattern was 10 units/hr for the first 5 minutes and then raised up to 20 units/hr, the new temporary basal rate would be 5 units/hr for the first 5 minutes and then raise up to 10 units/hr.

If the user has chosen a fixed percentage, he will be asked to choose a percent and to choose a period of time for the temporary basal rate. If the user chooses 50% and 1 hour, the basal rate will become 50% of the current basal rate and will remain at that rate for an hour. Using the example above, and assuming that the current basal rate is 10 units/hr, the basal rate will become 5 units/hr for the entire hour. It will not change after 5 minutes, when the original pattern would have increased.

In another embodiment of the invention, the actual basal rate is shown on the display screen. When the user scrolls up or down, the actual basal rate remains shown on the right hand side of the percentage inputted. When the user first enters a percentage or a different percentage, a prompt 530 may appear on the screen stating the calculated amount of insulin that the percentage is equivalent to in actual units per hour. For example, if 50% of the current basal rate is 10 units/hour, once the user inputs “50%,” a prompt will appear that states, “50% of the current basal rate is 10 units/hr. Go ahead?” The temporary basal rate will not be initiated until the user confirms 535.

The actual calculated dosage relieves the user of the need to calculate what the percentage selected of the current basal rate is equivalent to, providing a quick and accurate way to check the delivery dosage. After the user sees the actual value is that which was desired, when the percentage was entered, the user can confirm the selection. The feature also ensures that the percentage selected is appropriate and avoids delivering incorrect dosages. For example, the prompt may help the user realize if the user calculated the actual amount incorrectly and the percentage selected actually would deliver more or less than desired. The feature may also ensure that the user does not unknowingly enter the wrong percentage through a mis-typing.

In a further embodiment, the user may be notified at the end of the specified period for which the temporary basal rate is applied. At the end of that time, a prompt may alert the user and present a question on the display, asking whether the user wishes to continue the selected temporary basal rate, switch the format of the temporary basal rate being delivered, or confirming the return to the original basal rate. The feature may alert the user in any manner known in the art, such as for example, by a vibration or an audible alarm. The feature may be one that the user may initiate in a setting control, and can select to either activate or de-activate the feature.

While the description above refers to particular embodiments of the present invention, it will be understood that many modifications may be made without departing from the spirit thereof. The accompanying claims are intended to cover such modifications as would fall within the true scope and spirit of the present invention.

The presently disclosed embodiments are, therefore, to be considered in all respects as illustrative and not restrictive, the scope of the invention being indicated by the appended claims rather than the foregoing description. All changes that come within the meaning of and range of equivalency of the claims are intended to be embraced therein.

What is claimed is:

1. A method for temporarily adjusting a delivery rate of fluid from an infusion device, comprising:
prompting a user to select one of at least two options for a temporary basal rate type, the at least two options including a variable percentage of a basal rate and a fixed percentage of a basal rate, wherein the variable percentage remains at a substantially uniform percentage of the basal rate as it varies and the fixed percentage is a percentage of a constant value, the constant value being a current basal rate at or about the instant the fixed percentage is selected;
receiving into an infusion device the temporary basal rate type;
receiving into the infusion device a temporary basal rate in accordance to the temporary basal rate type; and
delivering a fluid at a delivery rate substantially equal to the temporary basal rate.

2. The method of claim 1, further including:
displaying a fluid dosage calculated from the temporary basal rate.

3. The method of claim 2, further including:
after displaying the fluid dosage and before delivering the fluid, prompting the user to confirm the temporary basal rate.

4. The method of claim 1, further including:
receiving into the infusion device a time period for delivery of the temporary basal rate.

5. The method of claim 4, wherein the time period is defined by a start time and an end time.

6. The method of claim 5, wherein the time period is defined by a start time and continues until the user inputs a command requesting the duration of time to end.

7. The method of claim 4, wherein the time period is defined by a start time immediately following confirmation of the temporary basal rate and an end time.

8. The method of claim 4, wherein the time period is defined by a duration of time, and the duration of time begins when the user inputs a command requesting the duration of time to begin.

9. The method of claim 1, wherein the infusion device includes a memory to store the temporary basal rate and the temporary basal rate type.

10. The method of claim 8, wherein the infusion device further includes a processor to retrieve the temporary basal rate and the temporary basal rate type from a memory and to control the delivery of the fluid according to the temporary basal rate and the temporary basal rate type.

11. The method of claim 1, wherein the fluid is insulin.

12. A method for temporarily adjusting the delivery rate of an infusion device, comprising:
inputting into an infusion device, configured to deliver a fluid at a basal rate, a selection of one of at least two options for a temporary basal rate type, the at least two options including a variable percentage of a basal rate and a fixed percentage of a basal rate, wherein the variable percentage remains at a substantially uniform percentage of the basal rate as it varies and the fixed percentage is a percentage of a constant value, the constant value being a current basal rate at or about the instant the fixed percentage is selected;
inputting a temporary basal rate in accordance to the temporary basal rate type into the infusion device; and
inputting an execution command into the infusion device, wherein in response to receiving the execution command the infusion device begin a program to deliver the fluid at a delivery rate substantially equal to the temporary basal rate.

13. The method of claim 12, further including:
inputting a time period for delivery of the temporary basal rate.

14. The method of claim 13, wherein the time period is defined by a start time and an end time.

15. The method of claim 14, wherein the time period is defined by a start time and continues until the user inputs a command requesting the duration of time to end.

16. The method of claim 13, wherein the time period is defined by a start time immediately following confirmation of the temporary basal rate and an end time.

17. The method of claim 13, wherein the time period is defined by a duration of time, and the duration of time begins when the user inputs a command requesting the duration of time to begin.

18. The method of claim 12, wherein the infusion device includes a memory to store the temporary basal rate and the temporary basal rate type.

19. The method of claim 18, wherein the infusion device further includes a processor to retrieve the temporary basal rate and the temporary basal rate type from the memory and to control the delivery of the fluid according to the temporary basal rate and the temporary basal rate type.

20. The method of claim 12, wherein the fluid is insulin.

21. A system for temporarily adjusting a delivery rate of fluid, comprising:
an infusion device including
a housing,
a reservoir in the housing that contains a fluid,
a drive system that forces the fluid from the reservoir, and
a user interface on the housing that prompts a user to select one of at least two options for a temporary basal rate type over a period of time, the at least two options including a variable percentage of a basal rate and a fixed percentage of a basal rate, wherein the variable percentage remains at a substantially uniform percentage of the basal rate as it varies and the fixed percentage is a percentage of a constant value, the constant value being a current basal rate at or about the instant the fixed percentage is selected;
a processor in communication with the drive system to regulate a rate at which the driving system forces the fluid from the reservoir by processing one or more basal patterns, the temporary basal rate, and the temporary basal rate type; and
a memory in communication with the processor to store the one or more basal patterns, the temporary basal rate, and the temporary basal rate type.

22. The system of claim 21, further including:
a display on the user interface of a fluid dosage calculated from the temporary basal rate.

23. The system of claim 22, further including:
a prompt to the user after displaying the fluid dosage, and before delivering the fluid, to confirm the temporary basal rate.

24. The system of claim 21, further including:
options for defining the period of time for delivery of the temporary basal rate, wherein the infusion device delivers the fluid at a delivery rate substantially equal to the temporary basal rate for the period of time.

25. The system of claim 24, wherein the time period is defined by a start time and an end time.

26. The system of claim 25, wherein the time period is defined by a start time and continues until the user inputs a command requesting the duration of time to end.

27. The system of claim 24, wherein the time period is defined by a start time immediately following confirmation of the temporary basal rate and an end time.

28. The system of claim 24, wherein the time period is defined by a duration of time, and the duration of time begins when the user inputs a command requesting the duration of time to begin.

29. The system of claim 21, wherein the fluid is insulin.

30. An infusion device for administration of a fluid, comprising:
a housing;
a reservoir in the housing that contains a fluid;
a drive system that forces the fluid from the reservoir; and
a user interface on the housing that prompts a user to select one of at least two options for a temporary basal rate type over a period of time, the at least two options including a variable percentage of a basal rate and a fixed percentage of a basal rate, wherein the variable percentage remains at a substantially uniform percentage of the basal rate as it varies and the fixed percentage is a percentage of a constant value, the constant value being a current basal rate at or about the instant the fixed percentage is selected.

31. The infusion device of claim 30, further including:
a processor in communication with the drive system to regulate a rate at which the driving system forces the fluid from the reservoir by processing one or more basal patterns, the temporary basal rate, and the temporary basal rate type.

32. The infusion device of claim 31, further including:
a memory in communication with the processor to store the one or more basal patterns, the temporary basal rate, and the temporary basal rate type.

33. The infusion device of claim 30, further including:
a display on the user interface of a fluid dosage calculated from the temporary basal rate.

34. The infusion device of claim 33, further including:
a prompt to the user after displaying the fluid dosage, and before delivering the fluid, to confirm the temporary basal rate.

35. The infusion device of claim 30, further including:
options for defining the period of time for delivery of the temporary basal rate, wherein the infusion device delivers the fluid at a delivery rate substantially equal to the temporary basal rate for the period of time.

36. The infusion device of claim 35, wherein the time period is defined by a start time and an end time.

37. The infusion device of claim 36, wherein the time period is defined by a start time and continues until the user inputs a command requesting the duration of time to end.

38. The infusion device of claim 35, wherein the time period is defined by a start time immediately following confirmation of the temporary basal rate and an end time.

39. The infusion device of claim 35, wherein the time period is defined by a duration of time, and the duration of time begins when the user inputs a command requesting the duration of time to begin.

40. The infusion device of claim 30, wherein the fluid is insulin.
